SEQUENCE LISTING (11 sequences)

SEQ ID NO: 1 - protein, 17 aa, synthetic construct
ALHNHYTQKSLSLSLGK

SEQ ID NO: 2 - protein, 17 aa, synthetic construct
ALHNRFTQKSLSLSPGK

SEQ ID NO: 3 - protein, 9 aa, synthetic construct
STYRVVSVL

SEQ ID NO: 4 - protein, 12 aa, synthetic construct
FLFPPKPKDTLM

SEQ ID NO: 5 - protein, 13 aa, synthetic construct
VMHEALHNHYTQK

SEQ ID NO: 6 - protein, 13 aa, synthetic construct
VMHEALHNRFTQK

SEQ ID NO: 7 - protein, 6 aa, synthetic construct
HNHYTQ

SEQ ID NO: 8 - protein, 6 aa, synthetic construct
HNRFTQ

SEQ ID NO: 9 - protein, 7 aa, synthetic construct
DTLMISR

SEQ ID NO: 10 - protein, 23 aa, synthetic construct
WQEGNVFSCSVMHEALHNHYTQK

SEQ ID NO: 11 - protein, 19 aa, synthetic construct
WQEGNVFSCSVMHEALHNR

(19) United States
(12) Patent Application Publication
(10) Pub. No.: US 2025/0076310 A1
(43) Pub. Date: Mar. 6, 2025

(54) METHODS TO CHARACTERIZING A FRAGMENT CRYSTALLIZABLE DOMAIN OF A BISPECIFIC ANTIBODY

(71) Applicant: Regeneron Pharmaceuticals, Inc., Tarrytown, NY (US)

(72) Inventors: Hui Xiao, Ridgewood, NJ (US); Bo Zhao, White Plains, NY (US); Yunlong Zhao, Scarsdale, NY (US); Ning Li, New Canaan, CT (US)

(21) Appl. No.: 18/528,268

(22) Filed: Dec. 4, 2023

Related U.S. Application Data
(60) Provisional application No. 63/434,516, filed on Dec. 22, 2022.
(60) Provisional application No. 63/431,131, filed on Dec. 8, 2022.

(51) Int. Cl. G01N 33/68; C12Q 1/37

(52) U.S. Cl. CPC G01N 33/6857; C12Q 1/37; G01N 33/6848; G01N 2440/38

(57) ABSTRACT

The present invention generally pertains to methods for characterizing the fragment crystallizable (Fc) region of a bispecific antibody (bsAb) using Hydrogen-Deuterium Exchange Mass Spectrometry (HDX-MS).

### CROSS-REFERENCE TO RELATED APPLICATIONS

This application incorporates by reference and claims priority to and the benefit of U.S. Provisional Patent Application No. 63/434,516, filed on Dec. 22, 2022 and U.S. Provisional Patent Application No. 63/431,131 filed on Dec. 8, 2022.

### REFERENCE TO SEQUENCE LISTING

This application includes a Sequence Listing submitted electronically as an XML file named 381206011SEQ, created Sep. 23, 2024, with a size of 10,633 bytes. The Sequence Listing is incorporated herein by reference.

### FIELD

The present invention generally pertains to methods for characterizing the fragment crystallizable (Fc) region of a bispecific antibody (bsAb) using Hydrogen-Deuterium Exchange Mass Spectrometry (HDX-MS).

### BACKGROUND

Bispecific antibodies hold great promise as therapeutic agents in a variety of indications. Bispecific antibodies with standard IgG composition can be difficult to produce because they contain four different polypeptide chains. The efficacy of smaller, more easily produced bispecific molecules has been clinically demonstrated in non-Hodgkin's lymphoma. See, for example, Bargou et al. (2008), Science 321(5891): 974-977. Likely due to the short in vivo half-life of this single chain molecule, daily administration was used to achieve these results. Therefore, there is a need in the art for bispecific therapeutic agents that have favorable pharmacokinetic properties along with therapeutic efficacy, case of administration, and configurations that simplify production.

Development of novel bispecific antibody (bsAb) platforms offers unprecedented opportunities for a wide variety of therapeutic applications. However, the expression and manufacturing of bsAbs with desired structures can be challenging. Owing to the uniqueness of each bsAb platform, more comprehensive and customized structural characterization is particularly important to understand the chemical or biological reactivity of bsAbs, as well as to guide process development, risk assessment and manufacturing. Therefore, it is important to develop reliable and accurate methods to support the development of novel bispecific antibodies.

### SUMMARY

Exemplary embodiments disclosed herein satisfy the aforementioned demands by providing methods for characterize the Fc region of a bispecific antibody.

In one exemplary embodiment, the method comprises incubating a sample comprising said bispecific antibody with a labeling buffer, contacting the labeled sample with a quenching buffer, contacting the quenched sample with a hydrolyzing agent to form a digested sample, contacting the digested sample to a liquid chromatography-mass spectrometer to determine a mass of deuterium-labeled peptides, and analyzing the mass of deuterium-labeled peptides to characterize the Fc domain.

In one aspect of this embodiment, the digested sample is desalted prior to contacting it to the liquid chromatography. The desalting can be performed on an immobilized column. One such column can be a SymmetryShield C8 trap column.

In one aspect of this embodiment, the characterizing comprises identifying site-specific mutations on the Fc domain. In another aspect of this embodiment, the characterizing comprises identifying a glycosylation profile of the Fc domain. In yet another aspect of this embodiment, the characterizing comprises identifying an oxidation profile of the Fc domain.

In one aspect of this embodiment, the analysis is performed by comparing said mass of deuterium-labeled peptides with masses of deuterium-labeled peptides obtained from characterizing a homodimer of a first heavy chain of the bispecific antibody and a homodimer the second heavy chain of the bispecific antibody obtained using the same or similar steps for characterization of a bispecific antibody described herein.

In one aspect of this embodiment, the hydrolyzing agent can be an agent capable of breaking bonds in a peptide or protein. In another aspect of this embodiment, the hydrolyzing agent can be pepsin. In another aspect of this embodiment, the hydrolyzing agent can be protease XIII. In yet another aspect of this embodiment, the hydrolyzing agent can be Aspergillus niger Prolyl Endo-protease.

In one aspect of this embodiment, the liquid chromatography is coupled to the mass spectrometer. In a specific aspect, the liquid chromatography is reversed-phase chromatography. In another specific aspect, the liquid chromatography can be performed at about 0° C.

In one aspect of this embodiment, a mobile phase used for the liquid chromatography of comprises formic acid in acetonitrile.

In one aspect of this embodiment, the hydrolyzing agent can be immobilized over a resin.

In one aspect of this embodiment, the labeling buffer comprises deuterated phosphate buffer. The labeling buffer can comprise a buffer containing deuterated water. The incubation with labeling buffer can be carried out by pulsed labeling, where the labeling time is constant, and samples also undergo a perturbation/equilibration step for various lengths of time. The incubation with labeling buffer can also be carried by continuous labeling, in which an identical equilibration step as pulsed labeling and a labeling step of variable length are performed.

In one aspect of this embodiment, the quenching buffer comprises sodium phosphate, guanidine hydrochloride, tris(2-carboxyethyl)phosphine, a reducing agent, or any combination thereof.

This disclosure also provides a method for identifying site-specific mutations on the Fc domain of a bispecific antibody.

In one exemplary embodiment, the method comprises incubating a sample comprising said bispecific antibody with a labeling buffer, contacting the labeled sample with a quenching buffer, contacting the quenched sample with a hydrolyzing agent to form a digested sample, contacting the digested sample to a liquid chromatography-mass spectrometer to determine a mass of deuterium-labeled peptides, and analyzing the mass of deuterium-labeled peptides to identify the site-specific mutations on the Fc domain.

In one aspect of this embodiment, the digested sample is desalted prior to contacting it to the liquid chromatography. The desalting can be performed on an immobilized column. One such column can be a SymmetryShield C8 trap column.

In one aspect of this embodiment, the analysis is performed by comparing said mass of deuterium-labeled peptides with masses of deuterium-labeled peptides obtained from characterizing a homodimer of a first heavy chain of the bispecific antibody and a homodimer the second heavy chain of the bispecific antibody obtained using the same or similar steps for characterization of a bispecific antibody described herein.

In one aspect of this embodiment, the hydrolyzing agent can be an agent capable of breaking bonds in a peptide or protein. In another aspect of this embodiment, the hydrolyzing agent can be pepsin. In another aspect of this embodiment, the hydrolyzing agent can be protease XIII. In yet another aspect of this embodiment, the hydrolyzing agent can be Aspergillus niger Prolyl Endo-protease.

In one aspect of this embodiment, the liquid chromatography is coupled to the mass spectrometer. In a specific aspect, the liquid chromatography is a reversed-phase chromatography step. In another specific aspect, the liquid chromatography can be carried out at or close to 0° C.

In one aspect of this embodiment, a mobile phase used for the liquid chromatography of comprises formic acid in acetonitrile.

In one aspect of this embodiment, the hydrolyzing agent can be immobilized over a resin.

In one aspect of this embodiment, the labeling buffer comprises deuterated phosphate buffer. The labeling buffer can comprise a buffer containing deuterated water. The incubation with labeling buffer can be carried out by pulsed labeling, where the labeling time is constant, and samples also undergo a perturbation/equilibration step for various lengths of time. The incubation with labeling buffer can also be carried by continuous labeling, in which an identical equilibration step as pulsed labeling and a labeling step of variable length are performed.

In one aspect of this embodiment, the quenching buffer comprises sodium phosphate, guanidine hydrochloride, tris(2-carboxyethyl)phosphine, a reducing agent, or any combination thereof.

This disclosure also provides a method for determining a glycosylation profile of the Fc domain a Fc domain of a bispecific antibody. In one exemplary embodiment, the method comprises incubating a sample comprising said bispecific antibody with a labeling buffer, contacting the labeled sample of with a quenching buffer, contacting the quenched sample with a hydrolyzing agent to form a digested sample, contacting the digested sample to a liquid chromatography-mass spectrometer to determine a mass of deuterium-labeled peptides, and analyzing the mass of deuterium-labeled peptides to determine the glycosylation profile the Fc domain.

In one aspect of this embodiment, the digested sample is desalted prior to contacting it to the liquid chromatography. The desalting can be performed on an immobilized column. One such column can be a SymmetryShield C8 trap column.

In one aspect of this embodiment, the characterizing comprises identifying site-specific mutations on the Fc domain. In another aspect of this embodiment, the characterizing comprises identifying a glycosylation profile of the Fc domain. In yet another aspect of this embodiment, the characterizing comprises identifying an oxidation profile of the Fc domain.

In one aspect of this embodiment, the hydrolyzing agent can be an agent capable of breaking bonds in a peptide or protein. In another aspect of this embodiment, the hydrolyzing agent can be pepsin. In another aspect of this embodiment, the hydrolyzing agent can be protease XIII. In yet another aspect of this embodiment, the hydrolyzing agent can be Aspergillus niger Prolyl Endo-protease.

In one aspect of this embodiment, the liquid chromatography is coupled to the mass spectrometer. In a specific aspect, the liquid chromatography is a reversed-phase chromatography step. In another specific aspect, the liquid chromatography can be carried out at or close to 0° C.

In one aspect of this embodiment, a mobile phase used for the liquid chromatography of comprises formic acid in acetonitrile.

In one aspect of this embodiment, the hydrolyzing agent can be immobilized over a resin.

In one aspect of this embodiment, the labeling buffer comprises deuterated phosphate buffer. The labeling buffer can comprise a buffer containing deuterated water. The incubation with labeling buffer can be carried out by pulsed labeling, where the labeling time is constant, and samples also undergo a perturbation/equilibration step for various lengths of time. The incubation with labeling buffer can also be carried by continuous labeling, in which an identical equilibration step as pulsed labeling and a labeling step of variable length are performed.

In one aspect of this embodiment, the quenching buffer comprises sodium phosphate, guanidine hydrochloride, tris(2-carboxyethyl)phosphine, a reducing agent, or any combination thereof.

### BRIEF DESCRIPTION OF THE DRAWINGS

FIG. 1 represents a schematic of an exemplary bispecific antibody format with site-specific mutations at the Fc domain of SEQ ID NO: 1 and SEQ ID NO:2.

FIG. 2 displays a hybrid significance testing of HDX-MS data for Fc-domain peptides including HC*/HC* homodimer vs. HC/HC homodimer, and HC/HC* heterodimer vs. HC/HC homodimer, according to an exemplary embodiment.

FIG. 3 is a series of panels, a-d. Panel a shows a structural homology of the Fc domain of bsAb-1 (SEQ ID NO:3, SEQ ID NO:4, SEQ ID NO:7, and SEQ ID NO:8), as characterized by an exemplary embodiment. Regions showing statistically significantly different deuterium uptake (HC*>HC) are in red. Regions with unchanged deuterium uptake after mutation are in gray. Panel b shows representative HDX-MS kinetic plots for C_{H}2 peptide FLFPPKPKDTLM (SEQ ID NO:4), as characterized by an exemplary embodiment. Panel c shows representative HDX-MS kinetic plots for C_{H}3 peptide VMHEALHNHYTQK (for HC) (SEQ ID NO:5) and VMHEALHNRFTQK (for HC*) (SEQ ID NO:6), as characterized by an exemplary embodiment. Panel d shows representative HDX-MS kinetic plots for C_{H}2 peptide STYRVVSVL (SEQ ID NO:3), as characterized by an exemplary embodiment.

FIG. 4 shows the relative abundance of glycosylation in HC/HC and HC*/HC* homodimers, and the HC/HC* heterodimer of bsAb-1, as characterized by an exemplary embodiment.

FIG. 5 is a series of panels a and b. Panel a displays deuterium uptake of FLFPPKPKDTLM (SEQ ID NO:4) in deglycosylated HC/HC and HC*/HC* homodimers, and the HC/HC* heterodimer of bsAb-1 with a 240 s labeling time, according to an exemplary embodiment. Panel b displays deuterium uptake of VMHEALHNHY(RF)TQK (SEQ ID NO:5 and SEQ ID NO:6) in deglycosylated HC/HC and HC*/HC* homodimers, and the HC/HC* heterodimer of bsAb-1 with a 240 s labeling time, according to an exemplary embodiment.

FIG. 6 is a series of panels a and b. Panel a shows quantification of HC Met²⁵⁶/HC* Met²⁵⁵ oxidation in bsAb-1 heterodimer and homodimers (SEQ ID NO:9), according to an exemplary embodiment. The levels of oxidation in 7 day and 14 day samples were normalized to those of the corresponding sample at day 0. Panel b shows quantification of HC Met⁴³²/HC* Met⁴³¹ oxidation in bsAb-1 heterodimer and homodimers (SEQ ID NO: 10 and SEQ ID NO:11), according to an exemplary embodiment. The levels of oxidation in 7 day and 14 day samples were normalized to those of the corresponding sample at day 0.

FIG. 7 is a series of panels, a-d. Panel a displays the significant differences in deuterium uptake for the C_{H}2 peptide FLFPPKPKDTLM (SEQ ID NO:4) between mAb 1-3 and bsAb-2-4, according to an exemplary embodiment. Panel b displays a normalized oxidation level of the C_{H}2 Met (SEQ ID NO:9) in heat stressed samples for mAb 1-3 and bsAb-2-4, according to an exemplary embodiment. Panel c displays the deuterium uptake of the C_{H}3 peptide VMHEALHNHYTQK (SEQ ID NO:5) with or without HY to RF mutations for mAb 1-3 and bsAb-2-4, according to an exemplary embodiment. Panel d displays the normalized oxidation level of the C_{H}3 Met (SEQ ID NO: 10 and SEQ ID NO: 11) in heat stressed samples for mAb 1-3 and bsAb-2-4, according to an exemplary embodiment.

FIG. 8 shows the deuterium uptake plot of the Fc domain peptides (SEQ ID NO:4, SEQ ID NO: 5, and SEQ ID NO:6) for mAbs 1-3 and bsAbs 1-4, according to an exemplary embodiment.

FIG. 9 shows a schematic representation of bio-layer interferometry assay used to study FcRn binding affinity to bsAb-1, according to an exemplary embodiment.

FIG. 10 shows DSC thermograms of a bispecific antibody in 1×PBS at a pH of 7.4, according to an exemplary embodiment. The black line represents the experimental data. The blue dotted lines represent the first thermal transition fits, and the green dotted lines represent the second thermal transition fits. The red line represents the cumulative fit data. A shows HC/HC homodimer. B shows HC/HC* heterodimer. C shows HC*/HC* homodimer.

### DETAILED DESCRIPTION

Bispecific antibodies (bsAbs), an emerging therapeutic modality, have greatly evolved over the past several decades and garnered tremendous interest for the treatment of numerous diseases, such as cancer, inflammatory disorders, autoimmunity and infectious diseases. See Brinkmann U, Kontermann R E, Bispecific antibodies: Bispecific antibodies have emerged as molecules with a multitude of talents. Science 2021; 372(6545):916-17; Labrijn A F, Janmaat M L, Reichert J M, Parren P W. Bispecific antibodies: a mechanistic review of the pipeline. Nature Reviews Drug discovery 2019; 18(8):585-608; Kufer P, Lutterbüse R, Bacuerle P A. A revival of bispecific antibodies. Trends in Biotechnology 2004; 22(5):238-44; Bargou R, Leo E, Zugmaier G, Klinger M, Goebeler M, Knop S, Noppency R, Viardot A, Hess G, Schuler M, Einsele H. Tumor regression in cancer patients by very low doses of a T cell-engaging antibody. Science 2008; 321(5891):974-77; Van Der Neut Kolfschoten M, Schuurman J, Losen M, Bleeker W K, Martínez-Martínez P, Vermeulen E, Den Bleker T H, Wiegman L, Vink T, Aarden L A, De Baets M H. Anti-inflammatory activity of human IgG4 antibodies by dynamic Fab arm exchange. Science 2007; 317(5844):1554-57; Van Roy M, Ververken C, Beirnaert E, Hoefman S, Kolkman J, Vierboom M, Breedveld E, t Hart B, Poelmans S, Bontinck L, Hemeryck A. The preclinical pharmacology of the high affinity anti-IL-6R Nanobody® ALX-0061 supports its clinical development in rheumatoid arthritis. Arthritis Research & Therapy 2015; 7(1):1-6 Khan S N, Sok D, Tran K, Movsesyan A, Dubrovskaya V, Burton D R, Wyatt R T. Targeting the HIV-1 spike and coreceptor with bi- and trispecific antibodies for single-component broad inhibition of entry. Journal of virology 2018; 92(18):e00384-18; and Brinkmann U, Kontermann R E. The making of bispecific antibodies. MAbs 2017; 9(2):182-212. However, generating bsAbs is challenging, because two asymmetric antigen-binding regions must be assembled with the desired configuration within the immunoglobulin (IgG) architecture. Recent developments in biochemical and genetic engineering techniques have opened a path to using a wide range of recombinant bsAb formats with unique physicochemical and biological properties depending on the molecular design. See id.

One format involves chemically crosslinking two antibodies or antibody subunits to form a desired configuration with bispecificity. See Ellerman D, Scheer J M. Generation of bispecific antibodies by chemical conjugation. Bispecific Antibodies 2011; 47-63. Springer, Berlin, Heidelberg; Doppalapudi V R, Huang J, Liu D, Jin P, Liu B, Li L, Desharnais J, Hagen C, Levin N J, Shields M J, Parish M. Chemical generation of bispecific antibodies. Proceedings of the National Academy of Sciences 2010; 107(52):22611-16. However, several limitations have been identified, including difficulties in manufacturing, proneness to aggregation, insufficient half-lives and potential safety concerns. An alternative design introduces “knobs into holes” mutations in the antibody Fc sequence; this scaffold enhances the desired heterodimerization rather than undesired mispairing. See Ridgway J B, Presta L G, Carter P. ‘Knobs-into-holes’ engineering of antibody C_{H}3 domains for heavy chain heterodimerization. Protein Engineering, Design and Selection 1996; 9(7):617-21; Spiess C, Bevers J, Jackman J, Chiang N, Nakamura G, Dillon M, Liu H, Molina P, Elliott J M, Shatz W, Scheer J M. Development of a human IgG4 bispecific antibody for dual targeting of interleukin-4 (IL-4) and interleukin-13 (IL-13) cytokines. Journal of Biological Chemistry 2013; 288(37):26583-93. Because this format retains Fc-mediated effector function, the half-life can be prolonged beyond those of non-Fc-containing counterparts. Nevertheless, the unnatural “knobs into holes” mutations may potentially induce immunogenicity or poor stability of the resulting bsAb, and therefore must be carefully evaluated during drug development and clinical studies. Furthermore, the removal of the two parental homodimers in the downstream purification process can be very difficult, because their properties are highly similar to those of the heterodimer.

Beyond approaches to promote heterodimerization, another strategy allows for selective purification of the heterodimers of interest from homodimer/heterodimer mixtures. In this format, mouse IgG2a and rat IgG2b antibodies are co-expressed within single cells. See Lindhofer H, Mocikat R, Steipe B, Thierfelder S. Preferential species-restricted heavy/light chain pairing in rat/mouse quadromas. Implications for a single-step purification of bispecific antibodies. The Journal of Immunology 1995; 155(1):219-25. Owing to the species specificity, each light chain (LC) can properly pair with its corresponding heavy chain (HC). Although two HCs can form dimers indiscriminately, heterodimers can be selectively purified from the mixtures through Protein A chromatography according to their differential binding affinities. However, the immunogenicity risk generated from the mouse IgG protein still poses challenges in the clinical application of this design. To address the aforementioned limitations of existing methods, the present invention was developed.

The present invention can help address the characterization of mutations that reside at the antibody C_{H}2-C_{H}3 interface. The antibody C_{H}2-C_{H}3 interface is a critical region that determines the Fc properties of antibodies. By comparing the deuterium uptake profiles of peptides from the bsAb heterodimer and two other corresponding homodimers (HC/HC and HC*/HC*), the present invention can identify potentially different folding structures at the region containing the site-specific mutations. The subsequent effects of these structural differences can also be evaluated, including Met oxidation near the mutation sites and the profiles of binding to FcRn. Thus, the present invention can improve fundamental understanding of the Fc properties of the bsAb format, and therefore provide guidance for process development, risk assessment and manufacturing.

Unless described otherwise, all technical and scientific terms used herein have the same meaning as commonly understood by one of ordinary skill in the art to which the present invention belongs. Although any methods and materials similar or equivalent to those described herein can be used in the practice or testing, particular methods and materials are now described. All publications mentioned are hereby incorporated by reference.

The term “a” should be understood to mean “at least one”; and the terms “about” and “approximately” should be understood to permit standard variation as would be understood by those of ordinary skill in the art; and where ranges are provided, endpoints are included.

The present invention includes a method to characterize a Fc domain of a bispecific antibody. As used herein, the term “bispecific antibody” (or bsAb) includes an antibody capable of selectively binding two or more epitopes. Bispecific antibodies generally comprise two different heavy chains with each heavy chain specifically binding a different epitope-either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or κλ-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310(2014), the entire teachings of which are herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology. Examples of bsAbs include those disclosed in the following patent applications, which are hereby incorporated by reference: U.S. Pat. No. 8,586,713; U.S. Publication No. 20130045492; U.S. Pat. Nos. 9,657,102; 10,626,142; 10,738,130; 10,772,972; U.S. Publication No. 20170174779; U.S. Publication No. 20170174781; U.S. Pat. No. 10,179,819 filed Jul. 29, 2016; and U.S. Pat. No. 11,142,578. Low levels of homodimer impurities can be present at several steps during the manufacturing of bispecific antibodies. The detection of such homodimer impurities can be challenging when performed using intact mass analysis due to low abundances of the homodimer impurities and the co-elution of these impurities with main species when carried out using a regular liquid chromatographic method.

The present invention can also be applicable to characterizing fragments of a multispecific antibody. As used herein “multispecific antibody” refers to an antibody with binding specificities for at least two different antigens. While such molecules normally will only bind two antigens (e.g., bispecific antibodies, bsAbs), antibodies with additional specificities such as trispecific antibody and KIH Trispecific can also be addressed by the system and method disclosed herein.

In some exemplary embodiments, the bispecific antibody can have a pI in the range of about 4.5 to about 9.0. In one exemplary specific embodiment, the pI can be about 4.5, about 5.0, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, or about 9.0. In some exemplary embodiments, the types of protein of interest in the compositions can be more than one.

In some exemplary embodiments, the bispecific antibody can be produced from mammalian cells. The mammalian cells can be of human origin or non-human origin can include primary epithelial cells (e.g., keratinocytes, cervical epithelial cells, bronchial epithelial cells, tracheal epithelial cells, kidney epithelial cells and retinal epithelial cells), established cell lines and their strains (e.g., 293 embryonic kidney cells, BHK cells, HeLa cervical epithelial cells and PER-C6 retinal cells, MDBK (NBL-1) cells, 911 cells, CRFK cells, MDCK cells, CHO cells, BeWo cells, Chang cells, Detroit 562 cells, HeLa 229 cells, HeLa S3 cells, Hep-2 cells, KB cells, LSI80 cells, LS174T cells, NCI-H-548 cells, RPMI2650 cells, SW-13 cells, T24 cells, WI-28 VA13, 2RA cells, WISH cells, BS-C-I cells, LLC-MK2 cells, Clone M-3 cells, 1-10 cells, RAG cells, TCMK-1 cells, Y-1 cells, LLC-PKi cells, PK (15) cells, GHi cells, GH3 cells, L2 cells, LLC-RC 256 cells, MHiCi cells, XC cells, MDOK cells, VSW cells, and TH-I, BI cells, BSC-1 cells, RAf cells, RK-cells, PK-15 cells or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, Midi cells, CHO cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C3H/IOTI/2 cells, HSDMiC3 cells, KLN205 cells, McCoy cells, Mouse L cells, Strain 2071 (Mouse L) cells, L-M strain (Mouse L) cells, L-MTK′ (Mouse L) cells, NCTC clones 2472 and 2555, SCC-PSA1 cells, Swiss/3T3 cells, Indian muntjac cells, SIRC cells, Cn cells, and Jensen cells, Sp2/0, NS0, NS1 cells or derivatives thereof).

As used herein, the “Fc region” refers to a region containing a fragment composed of a hinge portion or a part thereof, and CH2 and CH3 domains of an antibody molecule. An Fc region of IgG class means, for example, from cysteine at position 226 to the C terminus or from proline at position 230 to the C terminus according to Kabat's EU numbering (herein also referred to as EU index, but is not limited thereto. An Fc region may be obtained preferably by partially digesting IgG1, IgG2, IgG3, IgG4 monoclonal antibodies or such using a protease such as pepsin and then re-eluting a fraction adsorbed onto protein A column. The protease is not particularly limited as long as it can digest a full-length antibody so that Fab and F(ab′)2 will be produced in a restrictive manner by appropriately setting the enzyme reaction conditions such as pH, and examples include pepsin and papain.

The methods of present invention include incubating a sample comprising said bispecific antibody with a labeling buffer. The labeling buffer includes a buffer prepared in deuterated water.

The methods of present invention can include contacting a quenched sample with a hydrolyzing agent. As used herein, the term “hydrolyzing agent” refers to any one or combination of different agents that can perform digestion of a protein (enzymatically and non-enzymatically). Non-limiting examples of hydrolyzing agents that can carry out enzymatic digestion include protease from Aspergillus saitoi, elastase, subtilisin, protease XIII, pepsin, trypsin, Tryp-N, chymotrypsin, aspergillopepsin I, LysN protease (Lys-N), LysC endoproteinase (Lys-C), endoproteinas Asp-N (Asp-N), endoproteinasc Arg-C (Arg-C), endoproteinase Glu-C (Glu-C) or outer membrane protein T (OmpT), immunoglobulin-degrading enzyme of Streptococcus pyogenes (IdeS), thermolysin, papain, pronase, V8 protease or biologically active fragments or homologs thereof or combinations thereof. Non-limiting examples of non-enzymatic digestion include the use of high temperature, microwave, ultrasound, high pressure, infrared, solvents (non-limiting examples are ethanol and acetonitrile), immobilized enzyme digestion (IMER), magnetic particle immobilized enzymes, and on-chip immobilized enzymes. For a recent review discussing the available techniques for protein digestion, see Switzar et al., “Protein Digestion: An Overview of the Available Techniques and Recent Developments” (Linda Switzar, Martin Giera & Wilfried M. A. Niessen, Protein Digestion: An Overview of the Available Techniques and Recent Developments, 12 JOURNAL OF PROTEOME RESEARCH 1067-1077 (2013), the entire teachings of which are herein incorporated). One or a combination of hydrolyzing agents can cleave peptide bonds in a protein or polypeptide, in a sequence-specific manner, generating a predictable collection of shorter peptides. The ratio of hydrolyzing agent to protein and the time required for digestion can be appropriately selected to obtain optimal digestion of the protein. When the enzyme to substrate ratio (E/S) is unsuitably high, the correspondingly high digestion rate will not allow sufficient time for the peptides to be analyzed by mass spectrometer, and sequence coverage will be compromised. On the other hand, a low E/S ratio would need long digestion and thus long data acquisition time. The enzyme to substrate ratio can range from about 1:0.5 to about 1:200. As used herein, the term “digestion” refers to hydrolysis of one or more peptide bonds of a protein.

There are several approaches to carrying out digestion of a protein in a biological sample using an appropriate hydrolyzing agent, for example, enzymatic digestion or non-enzymatic digestion. One of the widely accepted methods for digestion of proteins in a sample involves the use of proteases. Many proteases are available and each of them have their own characteristics in terms of specificity, efficiency, and optimum digestion conditions. Proteases refer to both endopeptidases and exopeptidases, as classified based on the ability of the protease to cleave at non-terminal or terminal amino acids within a peptide. Alternatively, proteases also refer to the six distinct classes-aspartic, glutamic, and metalloproteases, cysteine, serine, and threonine proteases, as classified based on the mechanism of catalysis. The terms “protease” and “peptidase” are used interchangeably to refer to enzymes which hydrolyze peptide bonds. A person skilled in the art can choose an appropriate hydrolyzing agent for forming peptides to be analyzed using HDX-MS.

The methods of present invention include contacting a digested sample to a liquid chromatography-mass spectrometer to determine a mass of deuterium-labeled peptides.

As used herein, the term “liquid chromatography” refers to a process in which a biological/chemical mixture carried by a liquid can be separated into components as a result of differential distribution of the components as they flow through (or into) a stationary liquid or solid phase. Non-limiting examples of liquid chromatography include reverse phase liquid chromatography, ion-exchange chromatography, size exclusion chromatography, affinity chromatography, mixed-mode chromatography or hydrophobic chromatography.

As used herein, “affinity chromatography” can include separations including any method by which two substances are separated based upon their affinity to a chromatographic material. It can comprise subjecting the substances to a column comprising a suitable affinity chromatographic media. Non-limiting examples of such chromatographic media include, but are not limited to, Protein A resin, Protein G resin, affinity supports comprising an antigen against which a binding molecule (e.g., antibody) was produced, protein capable of binding to a protein of interest and affinity supports comprising an Fc binding protein. In one aspect, an affinity column can be equilibrated with a suitable buffer prior to sample loading. An example of a suitable buffer can be a Tris/NaCl buffer, pH around 7.0 to 8.0. A skilled artisan can develop a suitable buffer without undue burden. Following this equilibration, a sample can be loaded onto the column. Following the loading of the column, the column can be washed one or multiple times using, for example, the equilibrating buffer. Other washes, including washes employing different buffers, can be used before eluting the column. The affinity column can then be eluted using an appropriate elution buffer. An example of a suitable elution buffer can be an acetic acid/NaCl buffer, pH around 2.0 to 3.5. Again, the skilled artisan can develop an appropriate elution buffer without undue burden. The eluate can be monitored using techniques well known to those skilled in the art, including UV. For example, the absorbance at 280 nm can be employed, especially if the sample of interest comprises aromatic rings (e.g., proteins having aromatic amino acids like tryptophan).

As used herein, “ion exchange chromatography” can refer to separations including any method by which two substances are separated based on differences in their respective ionic charges, either on the molecule of interest and/or chromatographic material as a whole or locally on specific regions of the molecule of interest and/or chromatographic material, and thus can employ either cationic exchange material or anionic exchange material. Ion exchange chromatography separates molecules based on differences between the local charges of the molecules of interest and the local charges of the chromatographic material. A packed ion-exchange chromatography column or an ion-exchange membrane device can be operated in a bind-elute mode, a flowthrough mode, or a hybrid mode. After washing the column or the membrane device with an equilibration buffer or another buffer, product recovery can be achieved by increasing the ionic strength (i.e., conductivity) of the elution buffer to compete with the solute for the charged sites of the ion exchange matrix. Changing the pH and thereby altering the charge of the solute can be another way to achieve elution of the solute. The change in conductivity or pH may be gradual (gradient elution) or stepwise (step elution). Anionic or cationic substituents may be attached to matrices in order to form anionic or cationic supports for chromatography. Non-limiting examples of anionic exchange substituents include diethylaminoethyl (DEAE), quaternary aminoethyl (QAE) and quaternary amine (Q) groups. Cationic substituents include carboxymethyl (CM), sulfoethyl (SE), sulfopropyl (SP), phosphate (P) and sulfonate(S). Cellulose ion exchange medias or support can include DE23™, DE32™, DE52™, CM-23™, CM-32™, and CM-52™ are available from Whatman Ltd. Maidstone, Kent, U.K. SEPHADEX®-based and -locross-linked ion exchangers are also known. For example, DEAE-, QAE-, CM-, and SP-SEPHADEX® and DEAE-, Q-, CM- and S-SEPHAROSE® and SEPHAROSE® Fast Flow, and Capto™ S are all available from GE Healthcare. Further, both DEAE and CM derivitized ethylene glycol-methacrylate copolymer such as TOYOPEARL™ DEAE-650S or M and TOYOPEARL™ CM-650S or M are available from Toso Haas Co., Philadelphia, Pa., or Nuvia S and UNOSphere™ S from BioRad, Hercules, Calif., Eshmuno® S from EMD Millipore, MA.

As used herein, the term “hydrophobic interaction chromatography resin” can include a solid phase, which can be covalently modified with phenyl, octyl, butyl or the like. It can use the properties of hydrophobicity to separate molecules from one another. In this type of chromatography, hydrophobic groups such as, phenyl, octyl, hexyl or butyl can form the stationary phase of a column. Molecules such as proteins, peptides and the like pass through a HIC (hydrophobic interactive chromatography) column that possess one or more hydrophobic regions on their surface or have hydrophobic pockets and are able to interact with hydrophobic groups comprising a HIC's stationary phase. Examples of HIC resins or support include Phenyl sepharosc FF, Capto Phenyl (GE Healthcare, Uppsala, Sweden), Phenyl 650-M (Tosoh Bioscience, Tokyo, Japan) and Sartobind Phenyl (Sartorius corporation, New York, USA).

As used herein, the term “Mixed Mode Chromatography” or “multimodal chromatography” (both “MMC”) includes a chromatographic method in which solutes interact with a stationary phase through more than one interaction mode or mechanism. MMC can be used as an alternative or complementary tool to traditional reversed-phased (RP), ion exchange (IEX) and normal phase chromatography (NP). Unlike RP, NP and IEX chromatography, in which hydrophobic interaction, hydrophilic interaction and ionic interaction respectively are the dominant interaction modes, mixed-mode chromatography can employ a combination of two or more of these interaction modes. Mixed mode chromatography media can provide unique selectivity that cannot be reproduced by single mode chromatography. Mixed mode chromatography can also provide potential cost savings, longer column lifetimes and operation flexibility compared to affinity-based methods. In some exemplary embodiments, mixed mode chromatography media can be comprised of mixed mode ligands coupled to an organic or inorganic support, sometimes denoted a base matrix, directly or via a spacer. The support may be in the form of particles, such as essentially spherical particles, a monolith, filter, membrane, surface, capillaries, etc. In some exemplary embodiments, the support can be prepared from a native polymer such as cross-linked carbohydrate material, such as agarose, agPV, cellulose, dextran, chitosan, konjac, carrageenan, gellan, alginate, etc. To obtain high adsorption capacities, the support can be porous and ligands are then coupled to the external surfaces as well as to the pore surfaces. Such native polymer supports can be prepared according to standard methods, such as inverse suspension gelation (S Hjerten: Biochim Biophys Acta 79(2), 393-398 (1964), the entire teachings of which are herein incorporated). Alternatively, the support can be prepared from a synthetic polymer such as cross-linked synthetic polymers, for example, styrene or styrene derivatives, divinylbenzene, acrylamides, acrylate esters, methacrylate esters, vinyl esters, vinyl amides and the like. Such synthetic polymers can be produced according to standard methods, for example, “Styrene based polymer supports developed by suspension polymerization” (R Arshady: Chimica e L'Industria 70(9), 70-75(1988), the entire teachings of which are herein incorporated). Porous native or synthetic polymer supports are also available from commercial sources, such as such as GE Healthcare, Uppsala, Sweden.

As used herein, “reversed-phase chromatography” includes a chromatographic method wherein separation depends on the hydrophobic binding interaction between the solute molecule in the mobile phase and the immobilised hydrophobic ligand. The use of a hydrophobic stationary phase is essentially the reverse of normal phase chromatography, since the polarity of the mobile and stationary phases have been inverted—hence the term reversed-phase chromatography. Reversed-phase chromatography employs a polar (aqueous) mobile phase. As a result, hydrophobic molecules in the polar mobile phase tend to adsorb to the hydrophobic stationary phase, and hydrophilic molecules in the mobile phase will pass through the column and are eluted first. Hydrophobic molecules can be eluted from the column by decreasing the polarity of the mobile phase using an organic (non-polar) solvent, which reduces hydrophobic interactions. The more hydrophobic the molecule, the more strongly it will bind to the stationary phase, and the higher the concentration of organic solvent that will be required to elute the molecule.

The liquid chromatography step is generally carried out at about 0° C. The sub-zero degrees are applied with glycols administered into the buffer against freezing.

As used herein, the term “mass spectrometer” includes a device capable of identifying specific molecular species and measuring their accurate masses. The term is meant to include any molecular detector into which a polypeptide or peptide may be characterized. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends on the application. In some exemplary embodiments, the mass spectrometer can be a tandem mass spectrometer. As used herein, the term “tandem mass spectrometry” includes a technique where structural information on sample molecules is obtained by using multiple stages of mass selection and mass separation. A prerequisite is that the sample molecules be transformed into a gas phase and ionized so that fragments are formed in a predictable and controllable fashion after the first mass selection step. Multistage MS/MS, or MS^{a}, can be performed by first selecting and isolating a precursor ion (MS²), fragmenting it, isolating a primary fragment ion (MS³), fragmenting it, isolating a secondary fragment (MS⁴), and so on, as long as one can obtain meaningful information, or the fragment ion signal is detectable. Tandem MS has been successfully performed with a wide variety of analyzer combinations. Which analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time, mass spectrometer ions produced in the ion source can be trapped, isolated, fragmented, and m/z separated in the same physical device. The peptides identified by the mass spectrometer can be used as surrogate representatives of the intact protein and their post translational modifications. They can be used for protein characterization by correlating experimental and theoretical MS/MS data, the latter generated from possible peptides in a protein sequence database. The characterization includes, but is not limited, to sequencing amino acids of the protein fragments, determining protein sequencing, determining protein de novo sequencing, locating post-translational modifications, or identifying post translational modifications, or comparability analysis, or combinations thereof.

The present invention also includes that the analysis can be performed by comparing said mass of deuterium-labeled peptides with masses of deuterium-labeled peptides obtained from characterizing a homodimer of a first heavy chain of the bispecific antibody and a homodimer the second heavy chain of the bispecific antibody obtained using the same or similar steps for characterization of bispecific antibody described herein. Similarly, for a multispecific antibody, a comparison can be performed with antibody fragments that make up the multispecific antibody.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment comprises a sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

As used herein, the term “protein alkylating agent” refers to an agent used for alkylating certain free amino acid residues in a protein. Non-limiting examples of protein alkylating agents are iodoacetamide (IOA), chloroacetamide (CAA), acrylamide (AA), N-ethylmaleimide (NEM), methyl methanethiosulfonate (MMTS), and 4-vinylpyridine or combinations thereof.

The present invention can also include sample preparation including the use of a denaturing agent and/or a reducing agent.

As used herein, “protein denaturing” can refer to a process in which the three-dimensional shape of a molecule is changed from its native state. Protein denaturation can be carried out using a protein denaturing agent. Non-limiting examples of a protein denaturing agents include reducing agents, such as DTT or chaotropic agents. Protein denaturation can also be carried out by subjecting the protein to an increase in temperature, reducing the pH of the solution that contains the protein, or increasing the pH of the solution that contains the protein. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples for chaotropic agents include butanol, ethanol, guanidinium chloride, lithium perchlorate, lithium acetate, magnesium chloride, phenol, propanol, sodium dodecyl sulfate, thiourea, N-lauroylsarcosine, urea, and salts thereof.

As used herein, the term “protein reducing agent” refers to the agent used to reduce disulfide bridges in a protein. Non-limiting examples of protein reducing agents include dithiothreitol (DTT), β-mercaptoethanol, Ellman's reagent, hydroxylamine hydrochloride, sodium cyanoborohydride, tris(2-carboxyethyl)phosphine hydrochloride (TCEP-HCl), or combinations thereof.

The methods of present invention include contacting a labeled sample of with a quenching buffer. The quenching step can be performed in order to greatly decelerate the deuteration process in the protein via reducing the temperature and pH. In one embodiment, the target pH and temperature are ˜2.5 and <0° C., respectively. The quenching buffer can also contain a denaturing agent and a reducing agent to break the disulfide bonds. Non-limiting examples of denaturing agent and reducing agent are discussed previously herein.

Various publications, including patents, patent applications, published patent applications, accession numbers, technical articles and scholarly articles are cited throughout the specification. Each of these cited references is incorporated by reference, in its entirety.

The present invention will be more fully understood by reference to the following Examples. They should not, however, be construed as limiting the scope of the invention.

### EXAMPLES

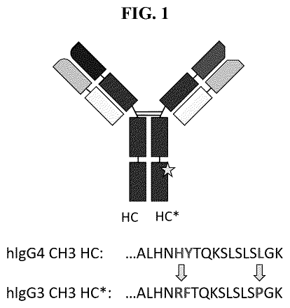

Materials. All reagents were commercially available and used as received unless stated otherwise. The chromatography solvents were of LC/MS grade and were obtained from Thermo Fisher Scientific (Waltham, MA). Antibodies (bsAbs 1-4 and mAbs 1-3) were produced by Regeneron (Tarrytown, NY). Deuterium oxide (D₂O), sodium phosphate, sodium chloride, guanidine hydrochloride, tris(2-carboxyethyl)-phosphine hydrochloride (TCEP-HCl), iodoacetamide and urea were purchased from Sigma-Aldrich (St. Louis, MO). The bsAbs tested were designed with an identical LC. See FIG. 1.

HDX-MS experiments. Labeling was performed on a LEAP PAL3 HDX automation system (Trajan Scientific and Medical, Morrisville, NC). Samples comprising 8 μL of 3 mg/mL mAb were labeled in 72 μL labeling buffer (1×PBS in D₂O, pH=7.4) three times for each indicated labeling time (30, 240, 1,800 and 14,400 s) at 25° C. After labeling, 50 μL of each labeled sample was quenched with 50 μL of quenching buffer (200 mM sodium phosphate, 4 M guanidine hydrochloride and 500 mM TCEP, pH=2.3, in water) at 1° C. After quenching, 50 μL of the sample was injected into a chromatography system within a cold box connected to a Waters Acquity UPLC instrument (Waters, Milford, MA). The cold box and LC solvent precooler were maintained at 0° C. for all experiments. Injected samples were passed over an immobilized pepsin column (2.1×30 mm, NovaBioAssays, Woburn, MA) at 100 μL/min for 120 s with mobile phase A (95% H₂O, 5% acetonitrile and 0.5% formic acid). The resulting peptic peptides were captured and desalted on a SymmetryShield C8 trap column (3.9×20 mm, Waters, Mildford, MA). The digested peptides were separated on an Acquity BEH C18 column (2.1×50 mm, 1.7 μm, 130 Å, Waters, Milford, MA) over a 25 min linear gradient of mobile phase B (0.1% formic acid in acetonitrile) from 0.1% to 30.0%. A Thermo Q Exactive™ Plus mass spectrometer was used to measure the mass of deuterium-labeled peptides.

HDX-MS data analysis. Raw files of both native and deuterated mAb samples were processed in the Protein Metrics Byos® with HDX workflow followed by in-house data refinement software that can select high quality peptide with a redundancy value close to 4. The refined list of peptides was manually validated in Protein Metrics software, and corrections to chromatographic peak integration limits were applied if necessary. After data processing, a “volcano plot” statistical testing was applied to identify peptides with significantly different deuterium uptake for each labeling time. See Hageman T S, Weis D D. Reliable identification of significant differences in differential hydrogen exchange-mass spectrometry measurements using a hybrid significance testing approach. Analytical Chemistry 2019; 91(13):8008-16.

Thermal stress and enzymatic digestion of mAbs. mAb drug substances were buffer exchanged to 10 mM histidine at pH=6.0, and then adjusted to 20 mg/mL for thermal stress. Samples were incubated at 45° C. for 1, 2 or 4 weeks. After thermal stress, each mAb sample was buffer exchanged to 5 mM acetic acid. Sample preparation for peptide mapping was implemented on a Beckman Coulter Biomek i5 automation system (Brea, CA). An aliquot of 100 μg of each sample was denatured and reduced in 40 μL solution containing 5 mM acetic acid and 5 mM TCEP-HCl by heating at 70° C. for 10 minutes. After cooling to room temperature, samples were adjusted to pH=8.0 by addition of 82 μL of H₂O and 24 μL of 1 M Tris-HCl. Each sample was then alkylated in the presence of 4 mM iodoacetamide and digested with trypsin at an enzyme-to-substrate ratio of 1:20 (w/w) at 30° C. for 140 minutes. After digestion, 56 μL of 8 M urea was added to the samples to maintain hydrophobic peptides in solution. Finally, 4.7 μL of 20% TFA was added to acidify the samples before MS analysis.

Peptide mapping and data analysis. For peptide mapping, peptide mixtures generated by trypsin digestion were separated with a Waters ACQUITY UPLC BEH C18 column (1.7 μm, 2.1 mm×150 mm) on a Waters ACQUITY UPLC system at a flow rate of 0.25 mL/minute. The column was equilibrated with 99.9% mobile phase A (0.05% TFA in water) before sample injection, with the column temperature maintained at 40° C. Subsequently, 5 μg of each tryptic digest sample was injected onto the column. Peptides were separated with a gradient profile held at 0.1% mobile phase B (0.045% TFA in acetonitrile) for the first 5 minutes, then increased from 0.1% to 40% mobile phase B over the next 80 minutes. A Thermo Q Exactive mass spectrometer was used for peptide mass analyses, and higher-energy collisional dissociation was used for peptide fragmentation for MS/MS experiments. Byonic™ was also used to aid in peptide identification. Skyline software was used for the extracted ion chromatogram peak area calculations. The relative abundance of a post translational modification (PTM) was determined by calculation of the ratio of the extracted ion chromatogram (EIC) peak area of the peptide containing the PTM to the sum of the EIC peak areas of the corresponding native peptide and the peptide containing the PTM.

Bio-layer interferometry. FcRn binding affinities to antibodies were determined through bio-layer interferometry. Biotinylated human FcRn receptor was captured on streptavidin-coated biosensor surfaces, and the biosensor was stabilized with binding buffer (100 mM sodium phosphate, 150 mM NaCl and 0.05% (v/v) surfactant PS20, pH=6.0). Subsequently, biotinylated human FcRn captured biosensors were dipped into assay plates containing antibody samples prepared at concentrations ranging from 2.5 μM to 0.078 UM, then shaken for 2 minutes at 1,000 rpm. The dissociation of bound antibody samples from the biosensors was conducted in binding buffer and monitored for 1 minute.

The sensorgram of a biosensor dipped in binding buffer in the absence of each antibody sample was subtracted from the binding sensorgrams to remove binding signal changes due to the dissociation of the captured biotinylated human FcRn receptor from the biosensors. Biosensors with no biotinylated human FcRn receptor captured were dipped into antibody samples prepared at concentrations ranging from 2.5 μM to 0.078 μM as a control. The sensorgrams were subtracted from the binding sensorgrams to remove binding signal changes due to non-specific binding to the biosensors. Owing to the rapid association and dissociation of antibodies to FcRn, the sensorgrams could not be fitted for kinetic analysis. Therefore, the resultant sensorgrams were subjected to steady state analysis, and the affinity constant (Kp) values were calculated.

### Example 1. Structural Differences Revealed by HDX-MS

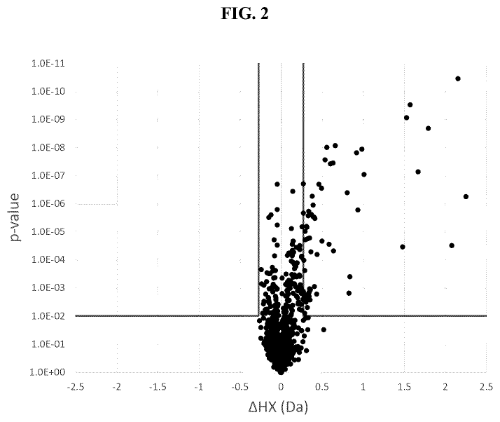

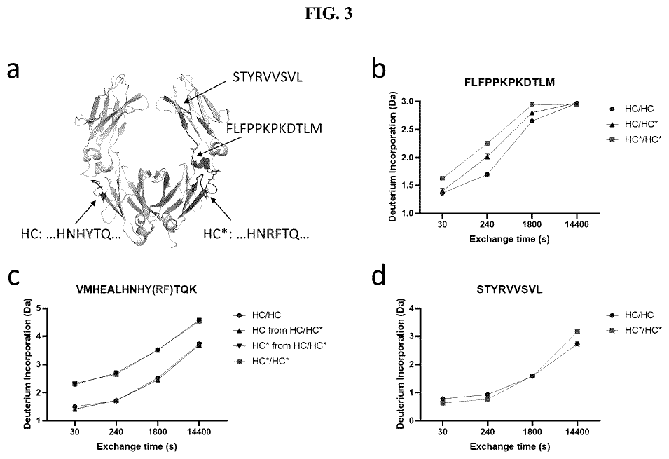

To evaluate the effects of site-specific mutations in the Fc domain, HDX-MS was performed on HC/HC* heterodimers of bsAb-1 and the two parental HC/HC and HC*/HC* homodimers. After pepsin digestion, 98.7%, 99.6% and 100% sequence coverage was achieved for HC, HC* and LC, respectively. Statistical testing was then performed to reveal the peptide sequences showing significantly different deuterium uptake profiles (FIG. 2). As shown in the homology model in FIG. 3, panel a, three regions showed significantly different deuterium uptake at the Fc domain in the HC* containing site-specific mutations (labeled with a side chain structure). The first region, with peptide sequence FLFPPKPKDTLM (SEQ ID NO:4), resides in the lower C_{H}2 region, which is in close contact with either the original His-Tyr or mutated Arg-Phe residues in the upper C_{H}3 region.

The HDX kinetic plot of this peptide is shown in FIG. 3, panel b. At earlier time points, deuterium uptake showed a trend of HC*/HC*>HC/HC*>HC/HC, whereas the uptake in all three molecules converged at later time points (e.g., 14,400 s). Of note, for the heterodimer containing HC and HC*, only half the peptide population was affected by the mutations. Interestingly, it was observed that the kinetics plot for the heterodimer indeed was intermediate between those of the two homodimers. Overall, these results indicated that the site-specific mutations might have led to increased dynamics of the FLFPPKPKDTLM (SEQ ID NO:4) peptide, without affecting the solvent accessibility or folding of this peptide.

The second region showing significant uptake differences is located at the C_{H}3 domain, which contains the mutated residues. Peptides with RF mutations (red trace) showed higher deuterium uptake than the original peptide with HY residues (blue trace) at all time points (FIG. 3, panel c). These findings may suggest that this peptide folds differently because of the mutations, thus increasing the structure's solvent exposure. Interestingly, the deuterium uptake profiles of these two heterodimeric peptides perfectly aligned with those of their corresponding homodimeric peptides, thus further validating the differences in local structure between HC and HC* within this asymmetric bsAb format.

According to the literature, the deuterium uptake of the aforementioned C_{H}2-C_{H}3 interface regions is sensitive to Fc glycosylation and Met oxidation. See Houde D, Peng Y, Berkowitz S A, Engen J R. Post-translational modifications differentially affect IgG1 conformation and receptor binding. Molecular & Cellular Proteomics 2010; 9(8):1716-28; Burkitt W, Domann P, O'Connor G. Conformational changes in oxidatively stressed monoclonal antibodies studied by hydrogen exchange mass spectrometry. Protein Science 2010; 19(4):826-35; Zhang A, Hu P, MacGregor P, Xue Y, Fan H, Suchecki P, Olszewski L, Liu A. Understanding the conformational impact of chemical modifications on monoclonal antibodies with diverse sequence variation using hydrogen/deuterium exchange mass spectrometry and structural modeling. Analytical Chemistry 2014; 86(7):3468-75; Wei B, Gao X, Cadang L, Izadi S, Liu P, Zhang H M, Hecht E, Shim J, Magill G, Pabon J R, Dai L. Fc galactosylation follows consecutive reaction kinetics and enhances immunoglobulin G hexamerization for complement activation. MAbs 2021; 13(1):1893427; and Kuhne F, Bonnington L, Malik S, Thomann M, Avenal C, Cymer F, Wegele H, Reusch D, Mormann M, Bulau P. The impact of immunoglobulin G1 Fc sialylation on backbone amide H/D exchange. Antibodies 2019; 8(4):49.

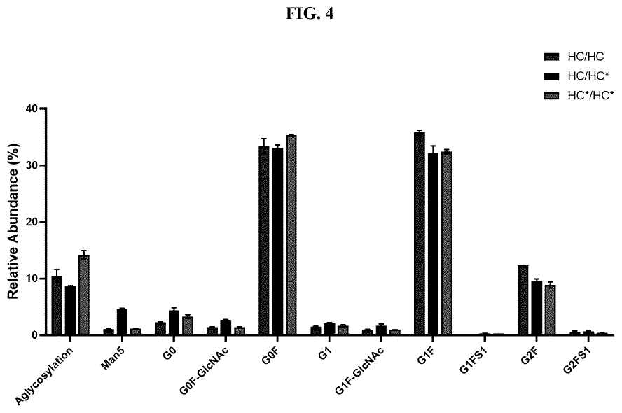

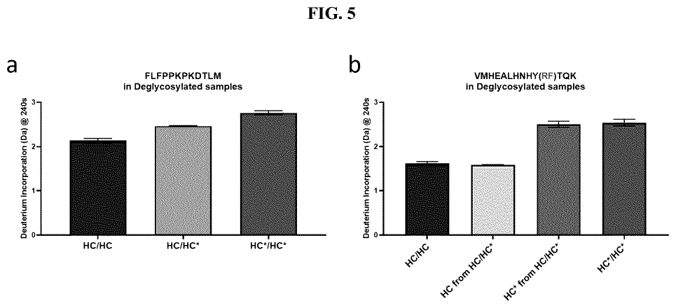

To confirm whether this structural difference was indeed caused by the mutations, the levels of glycosylation and Met oxidation of the three tested molecules were evaluated through reduced peptide mapping. Similar oxidation levels were observed for both Met at the C_{H}2-C_{H}3 interface (Table 1), thus ruling out the possibility of oxidation. Meanwhile, comparable glycosylation profiles were also obtained across all tested molecules, except that slightly larger differences (˜5%) were observed in the non-glycosylated form (FIG. 4). Lower glycan occupancy usually results in higher deuterium uptake for certain peptides that interact with glycan. The HC*/HC* homodimer showed higher levels of aglycosylation, thus possibly explaining the slightly higher level of uptake for peptides HC 302-310 next to the glycosylation site Asn³⁰¹ (FIG. 3, panel d). However, because higher aglycosylation contributed to the greater uptake for peptides in FIG. 3, panel b and panel c, HDX analysis was performed for all three deglycosylated molecules, in which the deuterium exchange was independent of the effects of glycans. As shown in FIG. 5, similar trends were observed in deglycosylated samples. Therefore, the structural deprotection identified in HC* appears to be caused primarily by the site-specific mutations.

TABLE 1
Level of Met oxidation in HC/HC and
HC*/HC* homodimers, and the HC/HC*
heterodimer of bsAb-1.

| | Oxidation (%) | | |
|---|---|---|---|
| | HC/HC | HC/HC* | HC*/HC |
| Peptides | homodimer | heterodimer | homodimer |
| DTLMISR | 3.8% | 5.4% | 4.1% |
| (SEQ ID | | | |
| NO: 9) | | | |
| WQEGNVFS | 1.7% | 1.9% | N/A |
| CSVMHEAL | | | |
| HNHYTQK | | | |
| (SEQ ID | | | |
| NO: 10) | | | |
| WQEGNVFS | N/A | 2.7% | 2.1% |
| CSVMHEAL | | | |
| HNR | | | |
| (SEQ ID | | | |
| NO: 11) | | | |

### Example 2. Effects of Structural Deprotection on Met Oxidation

The HDX analysis demonstrated that the C_{H}2-C_{H}3 interface of HC* exhibited structural deprotection, owing to the site-specific mutations. Consequently, it was hypothesized that structural deprotection might also render the two Met residues (HC Met²⁵⁶/HC* Met²⁵⁵ and HC Met⁴³²/HC* Met⁴³¹) located at the C_{H}2-C_{H}3 interface more prone to oxidation, thereby potentially affecting the shelf-life stability of bsAb. To test this hypothesis, the heterodimer and two homodimers of bsAb-1 were incubated at 45° C. for 7 days and 14 days, and quantified the relative abundance of oxidation on the basis of the peak areas of the EICs in reduced peptide mapping. The levels of Met oxidation at 7 days and 14 days were normalized to oxidation of the same Met site of each sample at 0 day; therefore, the rate of oxidation could be compared across three molecules.

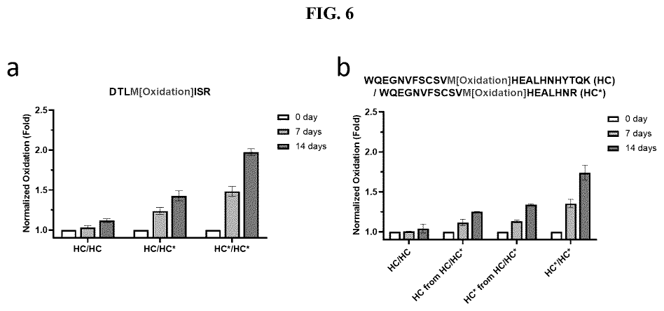

FIG. 6, panel a shows the rates of HC Met²⁵⁶/HC* Met²⁵⁵ oxidation, whose levels followed a trend of HC*/HC*>HC/HC*>HC/HC. Interestingly, the oxidation trend was positively correlated with the trend in backbone dynamics of the peptide containing this Met residue (FIG. 3, panel b), thus indicating that the Met within the higher dynamic environment was more prone to oxidation. As shown in FIG. 6, panel b, a similar correlation was also observed for the HC Met⁴³²/HC* Met⁴³¹ at the C_{H}2-C_{H}3 interface, with a rate of oxidation of HC/HC<HC*/HC* for the homodimers. The Met residue was more vulnerable to oxidation in structurally deprotected HC* than in HC from the same HC/HC* heterodimer (FIG. 3, panel c).

It was also observed that the rate of HC Met⁴³²/HC* Met⁴³¹ oxidation did not fully correlate with the deuterium uptake for this Met-containing peptide. For example, as revealed by HDX, the HC from both homodimers and heterodimers may possess the same folding structure and backbone dynamics (FIG. 3, panel c). However, HC Met⁴³² remained more susceptible to oxidation in the HC/HC* heterodimer than the HC/HC homodimer. Therefore, it was speculated that additional interactions might exist between HC and HC* within this asymmetric heterodimer, thus ultimately resulting in slightly different reactivity of the Met in the corresponding homodimer.

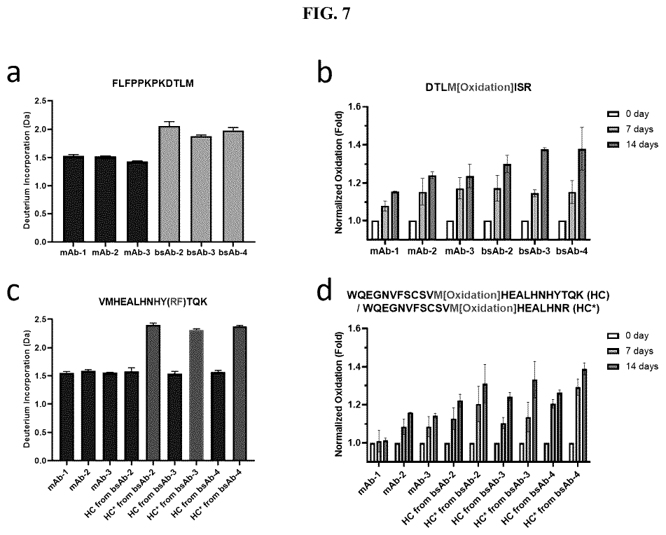

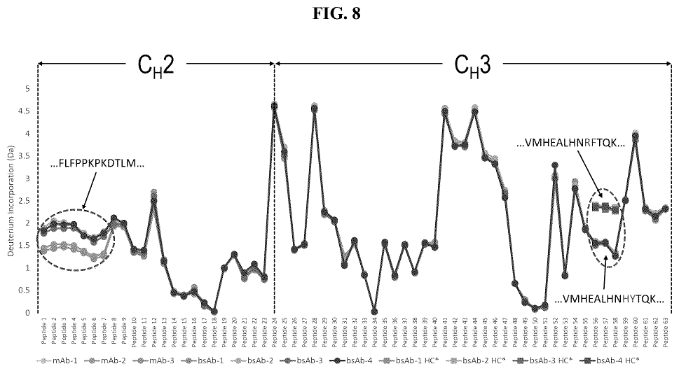

To confirm this observation, several other bsAbs with the same site-specific mutations, and similar glycosylation profiles and Met oxidation at TO were also examined (Table 2). Compared with regular IgG4 mAb 1-3, bsAb 2-4 all showed significantly higher deuterium uptake for both the peptides identified in bsAb-1 (FIG. 7, panel a and panel c), whereas the uptake was highly similar for all other peptides in the Fc domain (FIG. 8). The rates of Met oxidation were also evaluated for this collection of antibodies. A consistent correlation was observed in which bsAbs were more prone to oxidation of the two Met residues at the C_{H}2-C_{H}3 interface (FIG. 7, panel b and panel d). Overall, our results indicated that structural deprotection by Fc site-specific mutations rendered Met more vulnerable to oxidation at the C_{H}2-C_{H}3 interface; therefore, careful characterization is necessary during bsAb development and production.

TABLE 2
Levels of galactosylation, aglycosylation and Met oxidation at the
C_{H}2 peptide DTLMISR (SEQ ID NO: 9) in mAbs 1-3 and bsAbs 2-4.

| | | | Oxidation |
|---|---|---|---|
| | Galactosylation | Aglycosylation | at DTLMISR (SEQ |
| | (%) | (%) | ID NO: 9) (%) |
| mAb-1 | 22.3% | 3.7% | 2.4% |
| mAb-2 | 23.3% | 1.6% | 2.8% |
| mAb-3 | 33.0% | 4.7% | 2.6% |
| bsAb-2 | 19.1% | 3.3% | 3.3% |
| bsAb-3 | 27.3% | 9.7% | 3.6% |
| bsAb-4 | 16.6% | 4.1% | 3.0% |

### Example 3. Effects on FcRn Binding

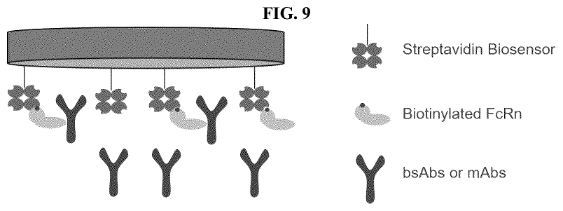

Numerous studies have demonstrated that the C_{H}2-C_{H}3 interface region participates in IgG Fc-FcRn binding, in which the strength of FcRn binding plays a crucial role in determining the serum half-life of therapeutic antibodies. See Roopenian D C, Akilesh S. FcRn: the neonatal Fc receptor comes of age. Nature reviews immunology. 2007; 7(9):715-25; Martin W L, West Jr A P, Gan L, Bjorkman P J. Crystal structure at 2.8 Å of an FcRn/heterodimeric Fc complex: mechanism of pH-dependent binding. Molecular cell. 2001; 7(4):867-77; Huang X, Zheng F, Zhan C G. Binding structures and energies of the human neonatal Fc receptor with human Fc and its mutants by molecular modeling and dynamics simulations. Molecular BioSystems. 2013; 9(12):3047-58; Bertolotti-Ciarlet A, Wang W, Lownes R, Pristatsky P, Fang Y, McKelvey T, Li Y, Li Y, Drummond J, Prueksaritanont T, Vlasak J. Impact of methionine oxidation on the binding of human IgG1 to FcRn and Fcγ receptors. Molecular immunology. 2009; 46(8-9):1878-82; and Mo J, Yan Q, So C K, Soden T, Lewis M J, Hu P. Understanding the impact of methionine oxidation on the biological functions of IgG1 antibodies using hydrogen/deuterium exchange mass spectrometry. Analytical Chemistry 2016; 88(19):9495-502. Because the bsAb format may fold asymmetrically at the C_{H}2-C_{H}3 interface region in HC and HC*, the FcRn binding profiles to both bsAb and regular IgG4 mAb were compared. Bio-layer interferometry was used to determine the FcRn binding affinities toward the HC/HC* homodimer and HC/HC* heterodimer of bsAb-1. In Table 3, highly similar K_{D} and maximum binding signal (Rₘₐₓ)ᵥₐₗᵤₑₛ were obtained for both samples. Therefore, both HC/HC and HC/HC* samples might have bound immobilized FcRn with 1:1 stoichiometry in our experimental setup (FIG. 9). On the basis of previous literature, FcRn may independently bind both sites of IgG Fc. See Abdiche Y N, Yeung Y A, Chaparro-Riggers J, Barman I, Strop P, Chin S M, Pham A, Bolton G, McDonough D, Lindquist K, Pons J. The neonatal Fc receptor (FcRn) binds independently to both sites of the IgG homodimer with identical affinity. MAbs 2015; 7(2):331-343. The HC with IgG4 sequence from bsAb might preferentially bind the FcRn. Therefore, the site-specific mutations in one half of the Fc have negligible effects on FcRn binding.

TABLE 3
Binding profiles of human FcRn to bsAb-1 samples.

| | Steady state analysis | | |
|---|---|---|---|
| Sample description | K_{D} (nM) | Rₘₐₓ (nm) | R² |
| HC/HC homodimer | 203 | 0.482 | 0.968 |
| HC/HC* heterodimer | 203 | 0.495 | 0.952 |

### Example 4. Effects on Fc Domain Stability

Capillary differential scanning calorimetry (DSC) data were collected to assess domain-dependent stability of bsAb-1 homodimers and heterodimer. All samples were diluted to 1 mg/mL in PBS, pH 7.4 and were performed in triplicates in the PEAQ-DSC instrument (MicroCal, Northampton, MA). All samples were subjected to a thermal ramp of 90° C./hr from 15° C. to 110° C. with a filtering period of 10 sec in “no feedback” mode. Each thermogram was background subtracted, fitted with a linear baseline, and analyzed using the PEAQ-DSC software. Melting temperatures were averaged and standard deviations were calculated from triplicate measurements.

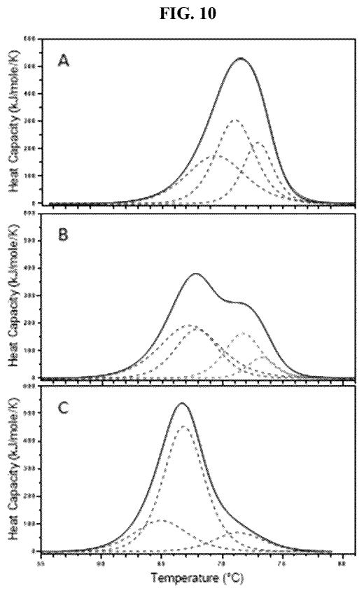

The structure of the C_{H}2-C_{H}3 interface becomes more flexible in HC*/HC* homodimer (with Fc mutations). Due to the Fc mutations, the domain specific stability was tested using DSC. FIG. 10 shows the C_{H}2 and C_{H}3 domain of HC/HC homodimer (panel A) shows higher melting temperatures than the HC*/HC* homodimer (panel C). The HC/HC* heterodimer (panel B) shows a mixture profile. This is consistent with observations using HDX-MS, wherein a more flexible structure leads to less domain stability.

What is claimed is:

1. A method of characterizing a Fc domain of a bispecific antibody, comprising:
a. incubating a sample comprising said bispecific antibody with a labeling buffer;
b. contacting the labeled sample with a quenching buffer;
c. contacting the quenched sample with a hydrolyzing agent to form a digested sample;
d. contacting the digested sample to a liquid chromatography-mass spectrometer to determine a mass of deuterium-labeled peptides; and
e. analyzing the mass of deuterium-labeled peptides to characterize the Fc domain.

2. The method of claim 1, wherein the digested sample is desalted prior to contacting it to the liquid chromatography.

3. The method of claim 1, wherein characterizing comprises identifying site-specific mutations on the Fc domain.

4. The method of claim 1, wherein characterizing comprises identifying a glycosylation profile of the Fc domain.

5. The method of claim 1, wherein characterizing comprises identifying an oxidation profile of the Fc domain.

6. The method of claim 1, wherein the analysis is performed by comparing said mass of deuterium-labeled peptides with masses of deuterium-labeled peptides obtained from characterizing a homodimer of the first heavy chain and a homodimer the second heavy chain using steps (a)-(e) of claim 1.

7. The method of claim 1, wherein the hydrolyzing agent is pepsin.

8. The method of claim 7, wherein characterizing comprises identifying site-specific mutations on the Fc domain.

9. The method of claim 7, wherein characterizing comprises identifying a glycosylation profile of the Fc domain.

10. The method of claim 1, wherein the liquid chromatography is coupled to the mass spectrometer.

11. The method of claim 1, wherein the hydrolyzing agent is immobilized over a resin.

12. The method of claim 1, wherein the labeling buffer comprises deuterated phosphate buffer.

13. The method of claim 1, wherein the quenching buffer comprises sodium phosphate, guanidine hydrochloride, tris(2-carboxyethyl)phosphine, a reducing agent, or any combination thereof.

14. The method of claim 1, wherein a mobile phase used for the liquid chromatography of step (d) comprises formic acid in acetonitrile.

15. A method of identifying site-specific mutations on Fc domain of a bispecific antibody with a first heavy chain and a second heavy chain, comprising:
a. incubating a first sample, a second sample and a third sample with a labelling buffer, wherein the first sample comprises said bispecific antibody, the second sample comprises a homodimer of the first heavy chain, and the third sample comprises a homodimer of the second heavy chain;
b. contacting each of the three labeled samples of with a quenching buffer;
c. contacting the quenched samples with a hydrolyzing agent to form a digested sample;
d. contacting the digested samples to a liquid chromatography-mass spectrometer to determine a mass of deuterium-labeled peptides; and
e. analyzing the mass of deuterium-labeled peptides to characterize the Fc domain by comparing said mass of deuterium-labeled peptides obtained using the first sample with masses of deuterium-labeled peptides obtained from the second sample and the third sample.

16. The method of claim 15, wherein the digested samples are desalted prior to contacting it to the liquid chromatography.

17. The method of claim 15, wherein the hydrolyzing agent is pepsin.

18. The method of claim 15, wherein the liquid chromatography is coupled to the mass spectrometer.

19. The method of claim 15, wherein the hydrolyzing agent is immobilized over a resin.

20. The method of claim 15, wherein the labeling buffer comprises deuterated phosphate buffer.

21. The method of claim 15, wherein the quenching buffer comprises sodium phosphate, guanidine hydrochloride, tris(2-carboxyethyl)phosphine, a reducing agent, or any combination thereof.

22. The method of claim 15, wherein a mobile phase used for the liquid chromatography of step (d) comprises formic acid in acetonitrile.
